SEQUENCE LISTING (4 sequences)

SEQ ID NO: 1 - DNA, 21 bp, Artificial Sequence
  primer
cggtcggtctggtcgttggtg

SEQ ID NO: 2 - DNA, 21 bp, Artificial Sequence
  primer
gtgctgtacatccggcccttt

SEQ ID NO: 3 - DNA, 21 bp, Artificial Sequence
  primer
cggtcggtctggtcgttggtg

SEQ ID NO: 4 - DNA, 21 bp, Artificial Sequence
  primer
gtgctgtacatccggcccttt

(19) United States
(12) Patent Application Publication
(10) Pub. No.: US 2009/0298138 A1
(43) Pub. Date: Dec. 3, 2009

(54) MICROORGANISM PRODUCING L-THREONINE HAVING AN INACTIVATED LYSR GENE, METHOD FOR PRODUCING THE SAME AND METHOD FOR PRODUCING L-THREONINE USING THE MICROORGANISM

(75) Inventors: Young-Hoon Park, Seongnam-city (KR); Byoung-Choon Lee, Seoul (KR); Sang-Jo Lim, Yongin-city (KR); Byoung-Hoon Kim, Incheon-city (KR); Sung-Soo Kim, Seoul (KR)

Correspondence Address: ROTHWELL, FIGG, ERNST & MANBECK, P.C., 1425 K STREET, N.W., SUITE 800, WASHINGTON, DC, 20005, US

(73) Assignee: CJ CORPORATION, Seoul (KR)

(21) Appl. No.: 11/720,906

(22) PCT Filed: Dec. 6, 2005
(86) PCT No.: PCT/KR05/04142

§ 371 Date: Jun. 5, 2007

(30) Foreign Application Priority Data
Dec. 6, 2004 (KR) 10-2004-0101664

(51) Int. Cl. C12P 13/08; C12N 1/21

(52) U.S. Cl. 435/115; 435/252.33

(57) ABSTRACT

Provided are a microorganism having an inactivated lysR gene in its chromosome and can produce L-threonine, a method of producing the microorganism, and a method d of producing L-threonine using the method. The microorganism can produce L-threonine with a high yield.

### TECHNICAL FIELD

The present invention relates to a microorganism having an inactivated lysR gene in its chromosome, a method of producing the microorganism, and a method of producing L-threonine using the microorganism.

### BACKGROUND ART

L-threonine is an essential amino acid and is widely used as a feed and food additive, and also as a pharmaceutical and raw material for synthesizing some drugs. It has been produced by fermentation with artificial mutants of the genus Escherichia, Coryneform bacteria, Seratia and Providencia. For example, Japanese Patent Publication No. 10037/81 discloses a method of producing L-threonine using a strain belonging to the genus Escherichia which has a nutritional requirement for diaminopimelic acid and methionine, and has the resistance to the feedback inhibition by threonine of the biosynthetic system of threonine. Japanese Patent Application Laid-open No. 224684/83 discloses a method of producing L-threonine using a strain belonging to the genus Brevibacterium which is resistant to S-(2-aminoethyl)-L-cysteine and α-amino-β-hydroxy valeric acid and has a nutritional requirement for L-isoleucine and L-lysine. Korean Patent Application Laid-open No. 8022/87 discloses a method of producing L-threonine using a strain which belongs to the genus Escherichia, requires diaminopimelic acid and methionine and is resistant to α-amino-β-hydroxy valeric acid, and which is additionally resistant to at least one of rifampicin, lysine, methionine, aspartic acid, and homoserine, or has a lower ability to decompose L-threonine. Japanese Patent Application Laid-open No. 219582/90 discloses a method of producing L-threonine using a strain that belongs to the genus Providencia, is resistant to α-amino-β-hydroxy valeric acid, L-ethionine, thiaisoleucine, oxythiamine, and sulfaguanidine, and has a requirement for L-leucine and a leaky requirement for L-isoleucine.

However, the above-known methods have the disadvantages that they fail to afford a high L-threonine productivity or require costly materials, such as diaminopimelic acid, isoleucine, etc. In other words, when diaminopimelic acid-requiring strains are used to produce L-threonine, an additional fermentation of diaminopimelic acid is required, thereby increasing costs. When an isoleucine-requiring strain is used to produce L-threonine, costly isoleucine must be added into a fermentation medium, thereby increasing costs.

In order to overcome these disadvantages, the present inventors developed a microorganism that can produce L-threonine with a higher yield than conventional strains through fermentation and has a leaky requirement for isoleucine, wherein there is no need to add isoleucine into a fermentation medium, and a strain requiring diaminopimelic acid, which is an intermediate involved in the synthesis of lysine, is not used. The L-threonine-producing microorganism belongs to Escherichia coli, has a nutritional requirement for methionine and a leaky requirement for isoleusine and is resistant to L-methionine analogues, L-threonine analogues, L-lysine analogues, and α-aminobutyric acid, and has a nutritional requirement for methionine and a leaky requirement for isoleucine, The L-threonine-producing microorganism and a method of producing L-threonine using the microorganism are patented (Korean Patent Publication No. 92-8365).

A LysR protein encoded by a conventional lysR gene represses the expression of lysC and lysA genes when the intracellular lysine concentration is high and increases the expression of lysC and lysA when the intracellular lysine concentration is low (Beacham, I. R., D. Hass, and E. Yagil. 1977. J. Bacteriol. 129:1034-1044). In E. coli K-12, lysC codes lysine-sensitive aspartokinase III (aspartate kinase III) [EC:2.7.2.4] that converts aspartic acid into aspartyl phosphate.

The present inventors have performed research intensively to screen a strain having a high L-threonine productivity based on the conventional techniques described above with the expectation that the rate of conversion of oxaloacetate obtained through the citric acid cycle into β-aspartyl phosphate, which refers to intermediates, such as lysine, threonine, methionine, etc., via aspartate and the yield of L-threonine will increase when the expression of lysC gene increases, found that the biosynthesis of L-threonine can be facilitated by inactivating the lysR gene, and completed the present invention.

### DETAILED DESCRIPTION OF THE INVENTION

### Technical Problem

The present invention provides a microorganism that can produce L-threonine with a higher yield.

The present invention also provides a method of producing the microorganism.

The present invention also provides a method of efficiently producing L-threonine using the microorganism.

### Technical Solution

According to an aspect of the present invention, there is provided a microorganism that can produce L-threonine and has an inactivated tyrR gene.

In the present invention, the microorganism can produce L-threonine and includes prokaryotic and eukaryotic microorganisms having an inactivated lysR gene. For example, strains belonging to the genus Escherichia, Erwinia, Serratia, Providencia, Corynebacterium and Brevibacterium can be included. Preferably, the microorganism belongs to Enterobacteriaceae family, and more preferably, to the genus Escherichia. Most preferably, the microorganism is Echerichia coli FTR7624 (KCCM-10538).

Examples of the microorganism having a lysR gene to be inactivated in the present invention include natural microorganisms and L-threonine-producing mutants. Examples of the mutants include microorganisms belonging to L-threonine-producing Escherichia coli which are resistant to L-methionine, L-threonine and L-lysine analogues and α-aminobutyric acid, and have a nutritional requirement for methionine and a leaky requirement for isoleucine; and microorganisms in which at least one copy of phosphoenol pyruvate carboxylase (ppc) gene and thrA, thrB, and thrC genes contained in a threonine operon is inserted in a chromosomal DNA, in addition to intrinsic ppc gene and genes in the threonine operon. The L-methionine analogue may be at least one compound selected from the group consisting of D,L-ethionine, norleucine, α-methylmethionine and L-methionine-D,L-sulfoxymine. The L-threonine analogue may be at least one compound selected from the group consisting of α-amino-β-hydroxy valeric acid and D,L-threonine hydroxamate. The L-lysine analogue may be at least one compound selected from the group consisting of S-(2-aminoethyl)-L-cysteine and λ-methyl-L-lysine. Other examples of the mutants include a microorganism in which a pckA gene involved in converting phosphoenol pyruvate (PEP) into oxaloacetate, which is an intermediate involved in the biosynthesis of L-threonine, is inactivated, a microorganism in which a tyrR gene repressing a lysC gene converting oxaloacetate into aspartate is inactivated, a microorganism in which a galR gene repressing the expression of a galP gene involved in the influx of glucose, etc.

In the present invention, the lysR gene encodes a protein regulating the express on of lysC gene at the transcription level thereby determines the level of aspartokinase activity in a cell. For Escherichia coli, the lysR gene is known and can be obtained from the genome sequence of E. coli published by Blattner et al. (Science 277:1453-1462 (1997)) (For example, accession no: EG10551). The genome sequence can also be obtained from National Center for Biotechnology Information (NCBI) in the U.S.A. and DNA Data Bank of Japan (DDBJ). The lysR gene according to the present invention also includes an allele generated due to the degeneracy of genetic code or a mutant which is functionally neutral. The term “inactivation” as used herein refers to a process of repressing the expression of an active lysR protein. For example, the inactivation can be inactivation induced by replacement, deletion, inversion, etc., of the lysR gene, inactivation induced by mutation in an expression regulating site of the lysR gene, and any process repressing the expression of the lysR gene.

Examples of the lysR gene to be inactivated in the present invention include, but are not limited to, lysR (Accession No. EG 10551) of E. coli K-12, lysR (Accession No. EG10551) of E. coli W3110, and lysR (Accession No. EG10551) of E. coli KCCM-10541.

The microorganism according to the present invention can be produced by inactivating a lysR gene present in a chromosome of a microorganism capable of producing L-threonine. The inactivation method may include causing mutation using light, such as UV rays, or chemicals and isolating strains having an inactivated lysR gene from the mutant. The inactivation method also includes a DNA recombination technology. The DNA recombination may be achieved, for example, by injecting a nucleotide sequence or vector including a nucleotide sequence with homology to the lysR gene into the microorganism to cause homologous recombination. The injected nucleotide sequence or vector may include a dominant selectable marker.

The present invention also provides a method of producing a L-threonine-producing microorganisim, including: preparing an inactivated lysR gene or a DNA fragment thereof; inserting the inactivated lysR gene or the DNA fragment thereof into a microorganism capable of producing L-threonine to cause recombination with a lysR gene present in a chromosome of the microorganism; and selecting the microorganism having the in activated lysR gene.

The “inactivated lysR gene or DNA fragment thereof” as used herein refers to a polynucleotide sequence that has a sequence homology to the lysR gene in a host but cannot express an active lysR protein due to a mutant caused by, for example, deletion, substitution, truncation, and inversion. The introduction of the inactivated lysR gene or DNA fragment thereof into a host cell can be achieved, for example, by transformation, conjugation, transduction, or electroporation, but is not limited thereto.

When the inactivated lysR gene or DNA fragment thereof is introduced into the host cell by transformation, the inactivation procedure can be carried out using a mixture of the polynucleotide sequence with a culture of the strain. In this case, although the strain is naturally competent to take up DNA and thus can be transformed, the strain may be previously rendered competent to take up DNA using any suitable method (See e.g. LeBlanc et al., Plasmid 28, 130-145, 1992; Pozzi et al., J. Bacteriol. 178, 6087-6090, 1996). The inactivated lysR gene or DNA fragment thereof is obtained by introducing a foreign DNA fragment into its genomic DNA fragment and replacing the wild-type chromosomal copy of the sequence with an inactive one. In an embodiment of the present invention, the inactivated polynucleotide sequence includes “tails” each containing a portion of DNA at a target site at the 5′ and 3′ ends thereof. The tails consists of at least 50 base pairs, preferably, greater than 200 to 500 base pairs for efficient recombination and/or gene conversion. For convenience, the inactivated polynucleotide sequence can include a selectable marker, for example, an antibiotic resistance gene. Where the target DNA is disrupted with an antibiotic resistance gene, transformants can be selected from an agar plate containing an appropriate antibiotic. Following transformation, the inactivated polynucleotide sequence introduced into the host cell undergoes homologous recombination with the genomic DNA tails, thereby inactivating the wild-type genomic sequence. Whether the inactivation recombination has occurred can be easily confirmed using, for example, Southern blotting, or polymerase chain reaction (PCR), which is a more convenient method.

A method of producing the L-threonine-producing microorganism according to an embodiment of the present invention includes the following procedures. First, a genomic DNA is isolated from a strain capable of producing L-threonine, and PCR is performed using the genomic DNA as a template according to a conventional technology to amplify the lysR gene. Next, the obtained lysR gene is cloned into a suitable plasmid or vector. The recombinant vector is introduced into a host cell such as E. coli through transduction. After the transformant is grown and cells are isolated, the recombinant vector having lysR gene is extracted. An antibiotic resistant gene fragment is then inserted into the lysR gene of the extracted recombinant vector to form a recombinant vector having an activated lysR gene. This recombinant vector is introduced into the host cell through transformation and cultured. Then, the propagated recombinant vector is isolated from the resultant transformant and treated with a suitable restriction enzyme to obtain a gene cassette including an inactivated lysR gene. Thereafter, the gene cassette is introduced into a strain capable of producing L-threonine using a conventional technique, such as electroporation, and microorganisms having an antibiotic resistance are selected to isolate microorganisms having an inactivated lysR gene.

It will be appreciated to those skilled in the art that the inactivated polynucleotide sequence according to the present invention can be easily obtained using a general cloning method. For example, a PCR amplification method using oligonucleotide primers targeting the lysR gene can be used.

In an embodiment of the present invention, recombinant plasmids pGemT/lysR and pGem/lysR::loxpCAT were constructed, and an inactivated gene cassette A lysR::loxpCAT was obtained therefrom. Then, an E. coli strain with Accession No. KCCM 10541 (FTR 2533) that is resistant to L-methionine, L-threonine and L-lysine analogues, has a nutritional requirement for methionine and a leaky requirement for isoleucine, and includes inactivated pckA, galR, and tyrR genes was transformed with the gene cassette using electroporation. As a result, the wild-type lysR gene is inactivated, thereby resulting one novel strain capable of producing a higher concentration of L-threonine than the parent strain. The novel strain was named E. coli FTR4014 and deposited with the Korean Culture Center of Microorganisms (KCCM) under the Budapest Treaty on Nov. 30, 2004 with Accession No. KCCM 10634.

E. coli FTR4014 according to the present invention was derived from E. coli with Accession No. KCCM 10541, which is a parent strain derived from E. coli with Accession No. KCCM 10236. E. coli KCCM 10236 was derived from E. coli KFCC 10718 (Korean Patent Publication No. 92-8365). E. coli KFCC 10718, which is a L-threonine-producing strain, has a nutritional requirement for methionine and is resistant to threonine analogues (for example, α-amino-β-hydroxy valeric acid, AHV), lysine analogues (for example, S-(2-aminoethyl-L-cysteine, AEC), isoleucine analogues (for example, α-aminobutyric acid), methionine analogues (for example, ethionine) and the like. The identified Korean Patent Publication is incorporated herein in its entirety by reference. Phosphoenol pyruvate (PEP) is a precursor of oxaloacetate which is an intermediate in a L-threonine biosynthesis pathway. E. coli Accession No. KCCM 10236 was obtained by inserting a ppc gene and a threonine operon (thr operon, thrABC), which were obtained from the chromosome of E. coli Accession No. KFCC 10718 capable of producing L-threonine, into the chromosome of the prototype E. coli Accession No. KFCC 10718 so that E. coli Accession No. KCCM 10236 includes two ppc genes and two threonine operons. E. coli Accession No. KCCM 10236 increases the expression of the ppc gene catalyzing the conversion of PEP into oxaloacetate, which is an intermediate involved in threonine biosynthesis, and genes (thrA: aspartokinase l-homoserine dehydrogenase, thrB: homoserine kinase, thrC: threonine synthase) involved in the synthesis of threonine from aspartate. E. coli FTR 4014 according to the present invention is derived from the parent strain E. coli KCCM 10541. E. coli KCCM 10541, which is obtained by specifically inactivating pckA and galR genes existing in the chromosome of E. coli KCCM 10236, increases the intracellular oxaloacetate concentration and the glucose influx rate, thereby increasing the yield of L-threonine and enabling high-speed fermentation. In addition, E. coli KCCM 10541, in which a tyrR gene existing in its chromosome is specifically inactivated, can increase the expression of a tyrB gene and the yield of L-threonone.

The present invention also provides a method of producing L-threonine, including: culturing a microorganism capable of producing L-threonine and having an inactivated lysR gene in its chromosome; and isolating L-threonine from the culture.

In the method of producing L-threonine according to the present invention, the culturing of the microorganism may be carried out in a suitable culture medium under suitable culturing conditions which are common in the art and can be easily adjusted according to the type of a selected strain by those skilled in the art. Examples of methods that can be used for the culturing include, but are not limited to, batch operation, continuous operation, fed-batch operation, etc.

L-threonine can be isolated from the culture using ordinary methods known in the art. Examples of isolations methods that can be used in the present invention include centrifugation, filtration, ion exchange chromatography, crystallization, etc. For example, L-threonine can be isolated using ion exchange chromatography from the supernatant obtained by centrifuging the culture at a low speed and removing biomass.

### Advantageous Effects

The microorganism according to the present invention having an inactivated lysR gene can produce L-threonine with a high yield through microbial fermentation

In addition, through the method of producing L-threonine according to the present invention, L-threonine can be produced with a high yield.

### DESCRIPTION OF THE DRAWINGS

FIG. 1 depicts the process of constructing recombinant plasmid pGemT/lysR; and

FIG. 2 depicts the process of obtaining DNA fragment A lysR::loxpCAT from the recombinant plasmid pGemT:lysR::loxpCAT.

### BEST MODE

Hereinafter, the present invention will be described in more detail with reference to the following examples. However, the following examples are provided only for illustrative purposes and are not intended to limited to the scope of the present invention.

### EXAMPLES

### Example 1

### Construction of Recombinant Plasmid and Knock-Out of lysR Gene

In the present Example, a lysR gene in a chromosome of E. coli was knocked-out through homologous recombination. To this end, a vector including a portion of the lysR gene was prepared and then transformed into E. coli host cell, followed by selecting strains having a knock-out lysR gene.

A genomic DNA was extracted from a wild type E. coli strain W3110 by using a QIAGEN Genomic-tip System. PCR was performed using the extracted genomic DNA as a template to obtain a DNA fragment of about 4 kb including the coding sequences of lysR and lysA genes. A portion of the coding sequence of the lysA gene was amplified through the PCR, thereby raising the probability of recombination in a subsequent recombination process. Oligonucleotides of SEQ ID NO. 1 and SEQ ID NO. 2 were used as primers. During the PCR, a cycle of denaturation at 94° C. for 30 seconds, annealing at 60° C. for 30 seconds, and extension at 72° C. for 4 minutes was repeated 30 times.

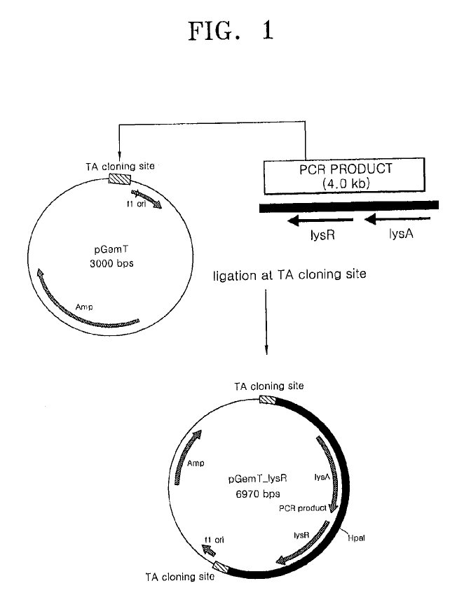

The PCR product was subjected to electrophoresis on a 0.8% agarose gel. DNA was purified from a band of 4078 bp. The purified DNA was ligated overnight to a TA site of a pGemT cloning vector (Promega Co.) at 16° C. to construct a recombinant plasmid pGemT/lysR (see FIG. 1). The resulting plasmid construct was transformed into E. coli DH5 α. The transformed strain was plated on a solid medium containing 50 mg/L of carbenicillin and cultured overnight at 37° C.

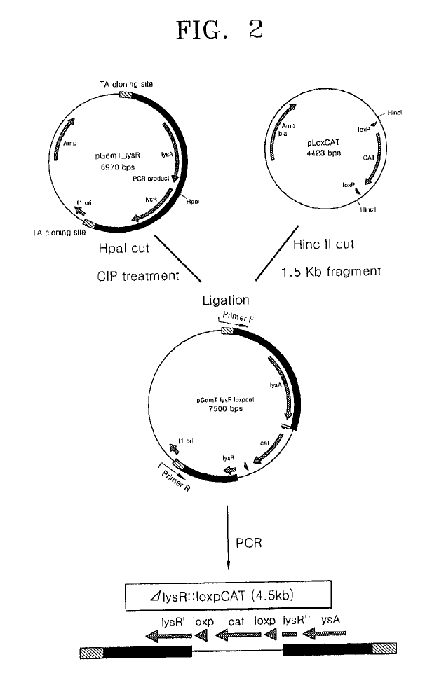

The obtained colonies were picked up with a platinum loop and inoculated into 3ml of a liquid LB medium containing carbenicillin. After overnight culturing, plasmid DNAs were extracted from the culture using a QIAGEN Mini Prep Kit (QIAGEN Co.). The plasmid DNA extract was digested with a restriction enzyme Hpa I and used to confirm whether a lysR gene had cloned. The confirmed plasmid pGemT/lysR was cleaved with the restriction enzyme Hpa I and loaded on a 0.8% agarose gel to separate DNA from a band of about 7.0 kb. A gene fragment of about 1.5 kb resistant to chloramphenicol that includes lox p sites, which were obtained by digesting plasmid pLoxCAT2 (Palmeros, B. et al, Gene 247 (1-2), 255-264, 2000) with Hinc II restriction enzyme, was constructed. Next, a Hpal fragment of plasmid pGemT/lysR and a Hindi fragment of plasmid pLoxCAT2 were ligated to obtain a recombinant plasmid pGemTAIysR::loxpCAT (about 7.5 kb) (refer to FIG. 2).

PCR was performed using the plasmid DNA as a template to amplify a DNA fragment (Δ lysR::loxpCAT) of about 4.6 kb including an ORF of lysR gene and loxpCAT sites. Oligonucleotides of SEQ ID NO. 1 and SEQ ID NO. 2 were used as primers. During the PCR, a cycle of denaturation at 94° C. for 30 seconds, annealing at 60° C. for 30 seconds and extension at 72° C. for 4 minutes was repeated 30 times.

### Example 2

### Knock-Out of lysR Gene of E.coli KCCM 10451 and Confirmation of Knock-Out of lysR Gene using PCR

The DNA fragment Δ lysR::loxpCAT constructed in Example 1 was transformed into L-threonine-producing E. coli strain of Accession No. KCCM 10541 (FTR2533) using electroporation and plated on a solid medium containing chloramphenicol to grow colonies having an inactivated lysR gene. PCR was performed to confirm whether the LysR gene had been specifically recombined in the screened candidate strain. Each of the parent strain KCCM 10541 and the candidate strain was cultured in a 3-mL of liquid medium overnight, and a genomic DNA was isolated from each of the cultures using a QIAGEN genomic kit 20.

PCR was performed using each of the genomic DNAs as a template to amplify a DNA fraction of about 4 kb or 5.5 kb including an ORF of the lysR gene or an ORF of the lysR gene and loxpCAT sites. Oligonucleotides of SEQ ID NO. 1 and SEQ ID NO. 2 were used as primers. During the PCR, a cycle of denaturation at 94° C. for 30 seconds, annealing at 60° C. for 30 seconds and extension at 72° C. for 4 minutes was repeated 30 times. When PCR was performed using the genomic DNA of the parent strain KCCM 10541 as a template, a DNA fragment of 4 kb was obtained. When PCR was performed using the genomic DNA of the candidate strain as a template, a DNA fragment of 5.5 kb including loxpCAT sites was obtained.

It was confirmed through the above-described process that the candidate strain contained a loxpCAT gene inactivating the lysR. The candidate strain was named E. coli FTR4014.

### Example 3

### Threonine Productivity of lysR-Inactivated Strain

E. coli FTR 4014 obtained in Example 2 was cultured in an Erlenmeyer flask containing a threonine titre medium having the composition in Table 1 below. The threonine productivity of the FTR 4014 strain was compared with that of the parent strain KCCM 10541.

TABLE 1
Threonine titer medium

| Components | Concentration (per liter) | |
|---|---|---|
| Glucose | 70 | g |
| Ammonium sulfate | 28 | g |
| KH₂PO₄ | 1.0 | g |
| MgSO₄•7H₂O | 0.5 | g |
| FeSO₄•7H₂O | 5 | mg |
| MnSO₄•8H₂O | 5 | mg |
| Calcium carbonate | 30 | g |
| L-methionine | 0.15 | g |
| Yeast extract | 2 | g |

pH 7.0

After the FTR 4014 strain was cultured on a LB solid medium overnight in an incubator at 32° C., one platinum loop of the culture was inoculated into 25 mL of the titer medium and cultured at 32° C. and 250 rpm for 48 hours. The results are shown in Table 2 below. Referring to FIG. 2, the threonine productivity of the parent strain KCCM 10541 is 23 g/L, and the threonine productivity of the recombinant strain FTR 4014 in which the lysR gene was inactivated is 25 g/L, indicating that there is an improvement of about 8.6% in productivity when the recombinant strain FTR 4014 is used.

TABLE 2
Results of flask titration test on recombinant strains

| | KCCM 10541 | FTR 4014 |
|---|---|---|
| Strain | (parent strain) | (variant strain) |
| L-threonine (g/L) | 23 | 25 |

The strain E. coli FTR 4014 was deposited with the Korean Culture Center of Microorganisms (KCCM) under the Budapest Treaty on Nov. 30, 2004 with Accession No. KCCM 10634.

While this invention has been particularly shown and described with reference to exemplary embodiments thereof, it will be understood by those skilled in the art that various changes in form and details may be made therein without departing from the spirit and scope of the invention as defined by the appended claims. The exemplary embodiments should be considered in descriptive sense only and not for purposes of limitation. Therefore, the scope of the invention is defined not by the detailed description of the invention but by the appended claims, and all differences within the scope will be construed as being included in the present invention.

1. An E. coli strain that has an inactivated lysR gene in a chromosome and can produce L-threonine.

2. The E. coli strain of claim 1 that has a nutritional requirement for methionine and is resistant to L-threonine analogues, L-lysine analogues, L-isoleucine analogues, and L-methionine analogues.

3. The E. coli strain of claim 1 being E. coli FTR 4014 of Accession No. KCCM 10634.

4. A method of producing a L-threonine-producing microorganisim, the method comprising:
preparing an inactivated lysR gene or a DNA fragment thereof;
introducing the inactivated lysR gene or the DNA fragment thereof into a microorganism capable of producing L-threonine to cause recombination with a lysR gene present in a chromosome of the microorganism; and
selecting the microorganism having the inactivated lysR gene.

5. The method of claim 4, wherein the inactivated lysR gene or the DNA fragment thereof is prepared by inserting a cassette containing an antibiotic marker (loxpKAN) into the lysR gene.

6. A method of producing L-threonine comprising: culturing the microorganism according to claim 1; and isolating L-threonine from the culture.
